(19) United States
(12) Patent Application Publication
(10) Pub. No.: US 2011/0184161 A1
(43) Pub. Date: Jul. 28, 2011

(54) USE OF MICROARRAYS FOR GENOMIC REPRESENTATION SELECTION

(76) Inventor: THOMAS J. ALBERT, Fitchburg, WI (US)

(21) Appl. No.: 13/084,259

(22) Filed: Apr. 11, 2011

Related U.S. Application Data
(63) Continuation of application No. 11/789,135, filed on Apr. 24, 2007, now abandoned.
(60) Provisional application No. 60/794,560, filed on Apr. 24, 2006.
(60) Provisional application No. 60/832,719, filed on Jul. 21, 2006.

(51) Int. Cl. C07H 21/04

(52) U.S. Cl. 536/23.1

(57) ABSTRACT

The present invention provides novel methods for reducing the complexity of a genomic sample for further analysis such as direct DNA sequencing, resequencing or SNP calling. The methods use pre-selected immobilized oligonucleotide probes to capture target nucleic acid molecules from a sample containing denatured, fragmented genomic nucleic acid. The disclosed method provides for cost-effective, flexible and rapid enrichment of target nucleic acid from complex biological samples.

### CROSS-REFERENCE TO RELATED APPLICATIONS

This application claims priority to co-pending U.S. Provisional Application No. 60/794,560, filed Apr. 24, 2006, and U.S. Provisional Application No. 60/832,719, filed Jul. 21, 2006, the disclosure of each of which is incorporated herein by reference in its entirety as if set forth herein.

### STATEMENT REGARDING FEDERALLY SPONSORED RESEARCH OR DEVELOPMENT

Not Applicable

### BACKGROUND OF THE INVENTION

The advent of DNA microarray technology makes it possible to build an array of millions of DNA sequences in a very small area, such as the size of a microscope slide. See, e.g., U.S. Pat. No. 6,375,903 and U.S. Pat. No. 5,143,854, each of which is hereby incorporated by reference in its entirety. The disclosure of U.S. Pat. No. 6,375,903 enables the construction of so-called maskless array synthesizer (MAS) instruments in which light is used to direct synthesis of the DNA sequences, the light direction being performed using a digital micromirror device (DMD). Using an MAS instrument, the selection of DNA sequences to be constructed in the microarray is under software control so that individually customized arrays can be built to order. In general, MAS-based DNA microarray synthesis technology allows for the parallel synthesis of over 4 million unique oligonucleotide features in a very small area of a standard microscope slide. The microarrays are generally synthesized by using light to direct which oligonucleotides are synthesized at specific locations on an array, these locations being called features.

With the availability of the entire genomes of hundreds of organisms, for which a reference sequence has generally been deposited into a public database, microarrays have been used to perform sequence analysis on DNA isolated from such organisms. DNA microarray technology has also been applied to many areas such as gene expression and discovery, mutation detection, allelic and evolutionary sequence comparison, genome mapping and more.

Many applications require searching for genetic variants and mutations across the entire human genome that underlie human diseases. In the case of complex diseases, these searches generally result in a single nucleotide polymorphism (SNP) or set of SNPs associated with disease risk. Identifying such SNPs has proved to be an arduous and frequently fruitless task because resequencing large regions of genomic DNA, usually greater than 100 kilobases (Kb) from affected individuals or tissue samples is frequently required to find a single base change or identify all sequence variants. Accordingly, the genome is typically too complex to be studied as a whole, and techniques must be used to reduce the complexity of the genome.

Therefore, alternative cost-effective and rapid methods for reducing the complexity of a genomic sample in a user defined way to allow for further processing and analysis would be a desirable contribution to the art.

### BRIEF SUMMARY OF THE INVENTION

The present invention is summarized as a novel method for reducing the complexity of a genomic sample to facilitate further processing and genetic analysis. The method uses pre-selected immobilized nucleic acid probes to capture target nucleic acid sequences from a genomic sample by hybridizing the sample to the probes on a substrate. The captured target genomic nucleic acids are washed and then eluted off of the substrate. The eluted genomic sequences are more amenable to detailed genetic analysis than a genomic sample that has not been subjected to this procedure. Accordingly, the disclosed method provides a cost-effective, flexible and efficient approach for reducing the complexity of a genomic sample.

In one aspect, the method provides a microarray having pre-selected array-immobilized nucleic acid probes to capture target nucleic acid sequences from a genomic sample. This may be accomplished by hybridizing the genomic sample of target nucleic acid sequence(s) against a microarray having array-immobilized nucleic acid probes directed to a specific region of the genome. After hybridization, target nucleic acid sequences present in the sample may be enriched by washing the array and eluting the hybridized genomic nucleic acids from the array. The target nucleic acid sequence(s), preferably DNA may be amplified using for example, non-specific ligation mediated PCR, resulting in an amplified pool of PCR products of reduced complexity compared to the original genomic sample.

In a related aspect, the invention provides a method of reducing the complexity of a genomic sample by hybridizing the sample against a microarray having array-immobilized pre-selected target nucleic acid probes under preferably stringent conditions sufficient to support hybridization between the array-immobilized probes and complementary regions of the genomic sample. The microarray is subsequently washed under conditions sufficient to remove non-specifically bound nucleic acids and the hybridized target genomic nucleic acid sequences are eluted from the microarray. The eluted target sequences may optionally be amplified.

In another aspect, the amplified target nucleic acid sequences may be sequenced, hybridized to a resequencing- or SNP-calling array and the sequence or genotypes may be further analyzed.

In another aspect, the invention provides an enrichment method for target nucleic acid sequences in a genomic sample, such as exons or variants, preferably SNP sites. This can be accomplished by programming genomic probes specific for a region of the genome to be synthesized on a microarray to capture complementary target nucleic acid sequences contained in a complex genomic sample.

Other objects, advantages and features of the present invention will become apparent from the following specification taken in conjunction with the accompanying drawings.

### BRIEF DESCRIPTION OF THE SEVERAL VIEWS OF THE DRAWINGS

FIG. 1 is a graphic depiction of flow diagram of the direct genomic selection process using a microarray.

FIG. 2 is a flow diagram of the direct genomic selection process using a microarray.

FIG. 3 diagrams a method for synthesizing probes on a microarray, releasing the probes from the microarray and immobilizing the probes on a support for use in a method for capturing target polynucleotides of interest.

### DETAILED DESCRIPTION OF THE INVENTION

The present invention broadly relates to cost-effective, flexible and rapid methods for reducing genomic sample complexity to enrich for target nucleic acids of interest and to facilitate further processing and analysis, such as sequencing, resequencing and SNP calling. The captured target nucleic acid sequences, which are of a more defined less complex genomic population are more amenable to detailed genetic analysis. Thus, the invention provides for method for enrichment of target nucleic acid in a complex genomic sample.

In one embodiment, a sample containing fragmented, denatured genomic nucleic acid molecules is exposed under hybridizing conditions to at least one oligonucleotide probe, and more typically a plurality of oligonucleotide probes, immobilized on a substrate to capture from the sample target nucleic acid molecules that hybridize to the immobilized probes. Non-hybridizing regions of the genome remain in solution. The probes correspond in sequence to at least one region of the genome and can be provided on a substrate in parallel using maskless array synthesis technology. Alternatively, probes can be obtained serially using a standard DNA synthesizer and then applied to the substrate. After the hybridization, nucleic acids that do not hybridize, or that hybridize non-specifically to the probes are separated from the substrate-bound probes by washing. The remaining nucleic acids, bound specifically to the probes, are eluted from the substrate in heated water or in a nucleic acid elution buffer to yield an eluate enriched for the target nucleic acid molecules.

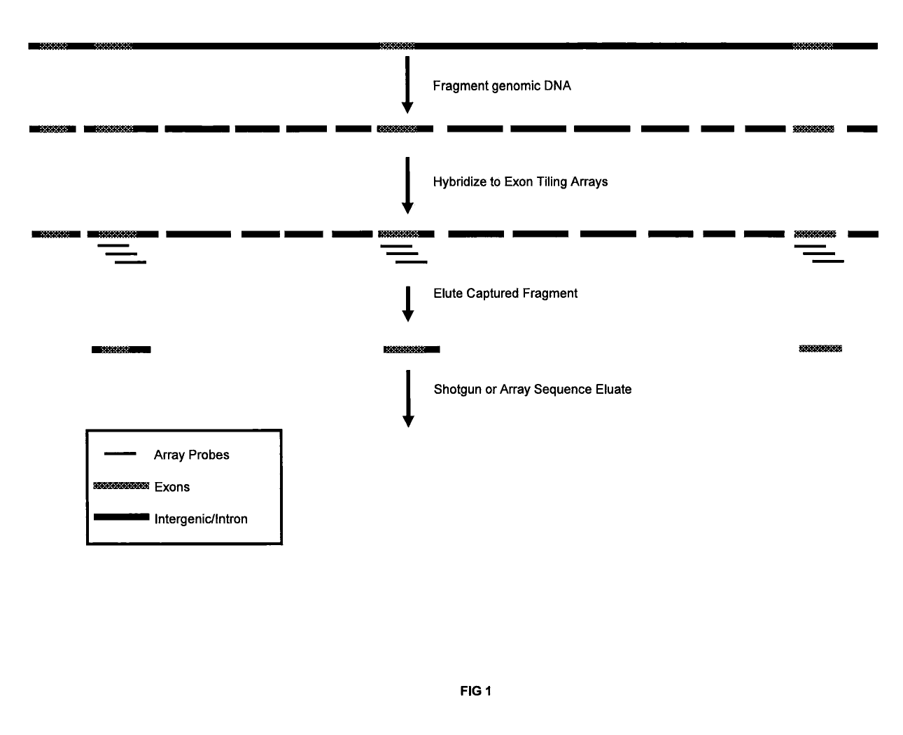

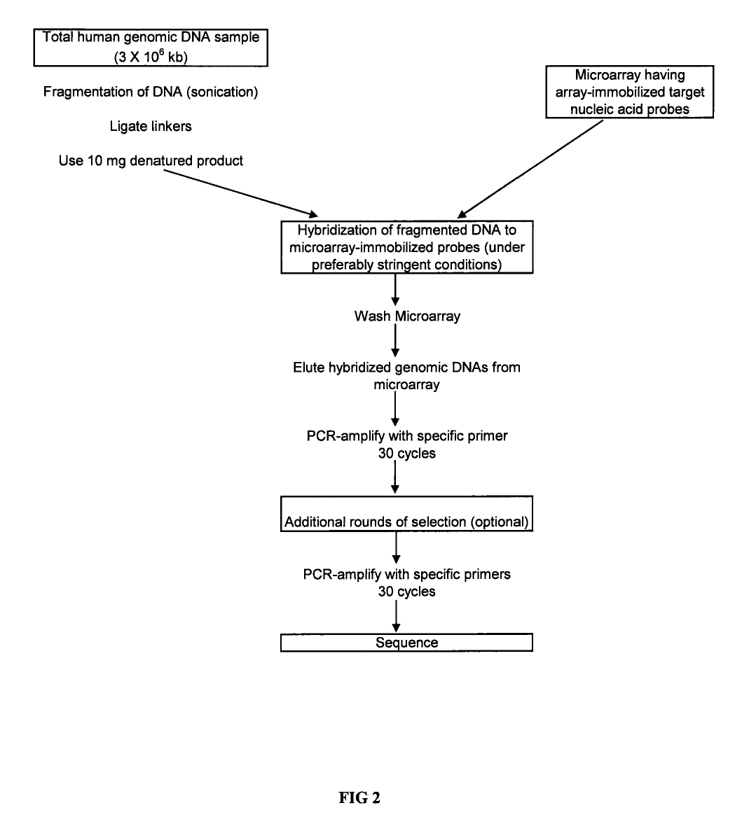

In some embodiments, double-stranded linkers are provided at the termini of the fragmented genomic nucleic acid before the fragments are denatured and hybridized to the immobilized probes. In such embodiments, target nucleic acid molecules can be amplified after elution to produce a pool of amplified products having reduced complexity relative to the original sample. The target nucleic acid molecules can be amplified using for example, non-specific ligation mediated PCR through multiple rounds of thermal cycling. Optionally, the amplified products can be further enriched by a second selection against the probes. The products of the second selection can be amplified again prior to use as described. This approach is summarized graphically in FIG. 1 and in a flow chart in FIG. 2.

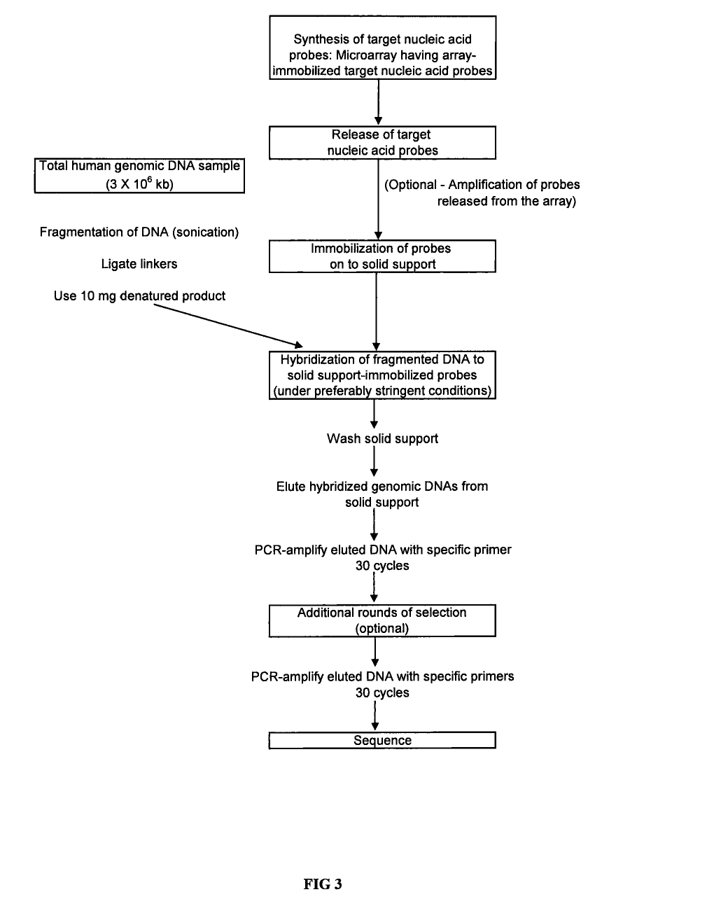

Alternatively, as shown in FIG. 3, nucleic acid probes for target molecules can be synthesized on a substrate, released from the substrate as a pool of probes and amplified as described. The amplified pool of released probes can be covalently- or non-covalently immobilized onto a support, such as glass, metal, ceramic or polymeric beads or other substrate. The probes can be designed for convenient release from the substrate by providing, e.g., at or near the substrate-proximal probe termini an acid- or alkali-labile nucleic acid sequence that releases the probes under conditions of low or high pH, respectively. Various cleavable linker chemistries are known in the art. The support can be provided, e.g., in a column having fluid inlet and outlet. The art is familiar with methods for immobilizing nucleic acids onto supports, for example by incorporating a biotinylated nucleotide into the probes and coating the support with streptavidin such that the coated support non-covalently attracts and immobilizes the probes in the pool. The sample or samples are passed across the probe-containing support under hybridizing conditions such that target nucleic molecules that hybridize to the immobilized support can be eluted for subsequent analysis or other use.

In one aspect, the invention enables capturing and enriching for target nucleic acid molecules or target genomic region(s) from a complex biological sample by direct genomic selection. The invention is also useful in searching for genetic variants and mutations, such as single nucleotide polymorphisms (SNP), or set of SNPs, that underlie human diseases. It is contemplated that capture and enrichment using microarray hybridization technology is much more flexible than other methods currently available in the field of genomic enrichment, such as use of BAC (bacterial artificial chromosome) for direct genomic selection (see Lovett et al. (1991) PNAS USA 88, 9628-9632).

The invention enables targeted array-based-, shotgun-, capillary-, or other sequencing methods known to the art. In general, strategies for shotgun sequencing of randomly generated fragments are cost-effective and readily integrated into a pipeline, but the invention enhances the efficiency of the shotgun approach by presenting only fragments from one or more genomic regions of interest for sequencing. The invention provides an ability to focus the sequencing strategies on specific genomic regions, such as such as individual chromosomes or exons for medical sequencing purposes.

Target nucleic acid molecules can be enriched from one or more samples that include nucleic acids from any source, in purified or unpurified form. The source need not contain a complete complement of genomic nucleic acid molecules from an organism. The sample, preferably from a biological source, includes, but is not limited to pooled isolates from individual patients, tissue samples, or cell culture. As used herein, the term “target nucleic acid molecules” refers to molecules from a target genomic region to be studied. The pre-selected probes determine the range of targeted nucleic acid molecules. The skilled person in possession of this disclosure will appreciate the complete range of possible targets and associated targets.

The target region can be one or more continuous blocks of several megabases, or several smaller contiguous or discontiguous regions such as all of the exons from one or more chromosomes, or sites known to contain SNPs. For example, the substrate can support a tiling array designed to capture one or more complete chromosomes, parts of one or more chromosomes, all exons, all exons from one or more chromosomes, selected exons, introns and exons for one or more genes, gene regulatory regions, and so on. Alternatively, to increase the likelihood that desired non-unique or difficult-to-capture targets are enriched, the probes can be directed to sequences associated with (e.g., on the same fragment as, but separate from) the actual target sequence, in which case genomic fragments containing both the desired target and associated sequences will be captured and enriched. The associated sequences can be adjacent or spaced apart from the target sequences, but the skilled person will appreciate that the closer the two portions are to one another, the more likely it will be that genomic fragments will contain both portions. Still further, to further reduce the limited impact of cross-hybridization by off-target molecules, thereby enhancing the integrity of the enrichment, sequential rounds of capture using distinct but related capture probe sets directed to the target region can be performed. Related probes are probes corresponding to regions in close proximity to one another in the genome that can, therefore, hybridize to the same genomic DNA fragment.

The nature and performance of the probes can be varied to advantageously adjust the distribution of the target molecules captured and enriched in accord with the methods. For example, the number of sequencing reactions required to effectively analyze each target region can be reduced by normalizing the number of copies of each target sequence in the enriched population such that across the set of probes the capture performance of distinct probes are normalized, on the basis of a combination of fitness and other probe attributes. Fitness, characterized by a “capture metric,” can be ascertained either informatically or empirically. In one approach, the ability of the target molecules to bind can be adjusted by providing so-called isothermal (Tm-balanced) oligonucleotide probes, as are described in U.S. Publication No. US-2005/0282209 (NimbleGen Systems, Madison, Wis.), that enable uniform probe performance, eliminate hybridization artifacts and/or bias and provide higher quality output. Probe lengths are adjusted (typically, 45 mer-85 mer but optionally more than 100 nt) to equalize the melting temperature (Tm=76° C.) across the entire set. Thus, probes are optimized to perform equivalently at a given stringency in the genomic regions of interest, including AT- and GC-rich regions. Relatedly, the sequence of individual probes can be adjusted, using natural bases or synthetic base analogs such as inositol, or a combination thereof to achieve a desired capture fitness of those probes. Similarly, locked nucleic acid probes, peptide nucleic acid probes or the like having structures that yield desired capture performance can be employed. The skilled artisan in possession of this disclosure will appreciate that probe length, melting temperature and sequence can be coordinately adjusted for any given probe to arrive at a desired capture performance for the probe.

Capture performance can also be normalized by ascertaining the capture fitness of probes in the probe set, and then adjusting the quantity of individual probes on the substrate accordingly. For example, if a first probe captures twenty times as much nucleic acid as a second probe, then the capture performance of both probes can be equalized by providing twenty times as many copies of the second probe, for example by increasing by twenty-fold the number of features displaying the second probe. If the probes are prepared serially and applied to the substrate, the concentration of individual probes in the pool can be varied in the same way.

Still further, another strategy for normalizing capture of target nucleic acids is to subject the eluted target molecules to a second round of hybridization against the probes under less stringent conditions than were used for the first hybridization round. Apart from the substantial enrichment in the first hybridization that reduces complexity relative to the original genomic nucleic acid, the second hybridization can be conducted under hybridization conditions that saturate all capture probes. Presuming that substantially equal amounts of the capture probes are provided on the substrate, saturation of the probes will ensure that substantially equal amounts of each target are eluted after the second hybridization and washing.

Another normalizing strategy follows the elution and amplification of captured target molecules from the substrate. Target molecules in the eluate are denatured to a single-stranded state and are re-annealed. Kinetic considerations dictate that abundant species re-anneal before less abundant species. As such, by removing the initial fraction of re-annealed species, the remaining single-stranded species will be balanced relative to the initial population in the eluate. The timing required for optimal removal of abundant species is determined empirically.

As used herein, the term “hybridization” is used in reference to the pairing of complementary nucleic acids. Hybridization and the strength of hybridization (i.e., the strength of the association between the nucleic acids) is affected by such factors as the degree of complementary between the nucleic acids, stringency of the conditions involved, the Tₘ of the formed hybrid, and the G:C ratio of the nucleic acids. While the invention is not limited to a particular set of hybridization conditions, stringent hybridization conditions are preferably employed. Stringent hybridization conditions are sequence-dependent and will differ with varying environmental parameters (e.g., salt concentrations, and presence of organics). Generally, “stringent” conditions are selected to be about 5° C. to 20° C. lower than the thermal melting point (Tm) for the specific nucleic acid sequence at a defined ionic strength and pH. Preferably, stringent conditions are about 5° C. to 10° C. lower than the thermal melting point for a specific nucleic acid bound to a complementary nucleic acid. The Tm is the temperature (under defined ionic strength and pH) at which 50% of a nucleic acid (e.g., tag nucleic acid) hybridizes to a perfectly matched probe.

Similarly, “stringent” wash conditions are ordinarily determined empirically for hybridization of each set of tags to a corresponding probe array. The arrays are first hybridized (typically under stringent hybridization conditions) and then washed with buffers containing successively lower concentrations of salts, or higher concentrations of detergents, or at increasing temperatures until the signal-to-noise ratio for specific to non-specific hybridization is high enough to facilitate detection of specific hybridization. Stringent temperature conditions will usually include temperatures in excess of about 30° C., more usually in excess of about 37° C., and occasionally in excess of about 45° C. Stringent salt conditions will ordinarily be less than about 1000 mM, usually less than about 500 mM, more usually less than about 150 mM. However, the combination of parameters is more important than the measure of any single parameter. See, e.g., Wetmur et al., J. Mol. Biol. 31:349-70 (1966), and Wetmur, Critical Reviews in Biochemistry and Molecular Biology 26(34):227-59 (1991).

“Stringent conditions” or “high stringency conditions,” as defined herein, can be hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 mg/ml), 0.1% SDS, and 10% dextran sulfate at 42° C., with washes at 42° C. in 0.2×SSC (sodium chloride/sodium citrate) and 50% formamide at 55° C., followed by a wash with 0.1×SSC containing EDTA at 55° C.

By way of example, but not limitation, it is contemplated that buffers containing 35% formamide, 5×SSC, and 0.1% (w/v) sodium dodecyl sulfate are suitable for hybridizing under moderately non-stringent conditions at 45° C. for 16-72 hours. Furthermore, it is envisioned that the formamide concentration may be suitably adjusted between a range of 20-45% depending on the probe length and the level of stringency desired. Also encompassed within the scope of the invention is that probe optimization can be obtained for longer probes (>>50 mer), by increasing the hybridization temperature or the formamide concentration to compensate for a change in the probe length. Additional examples of hybridization conditions are provided in several sources, including: “Direct selection of cDNAs with large genomic DNA clones,” in Molecular Cloning: A Laboratory Manual (eds. Sambrook, J. & Russell, D. W.) Chapter 11 Protocol 4, pages 11.98-11.106 (Cold Spring Harbor Laboratory Press, Cold Spring Harbor, N.Y., USA, 2001).

The following examples are provided as further non-limiting illustrations of particular embodiments of the invention.

### EXAMPLES

### Example 1

This example describes modifications to direct selection that allow for rapid and efficient discovery of new polymorphisms and mutations in large genomic regions. Microarrays having immobilized probes were used in one- or multiple rounds of hybridization selection with a target of total genomic DNA, and the selected sequences were amplified by the polymerase chain reaction (PCR) (see FIGS. 1 and 2).

Preparation of the Genomic DNA and Double-Stranded Linkers

DNA was fragmented using sonication to an average size of ˜500 base pairs.

A reaction to polish the ends of the sonicated DNA fragments was set up:

| | |
|---|---|
| DNA fragments | 41 microliters |
| T4 DNA Polymerase | 20 microliters |
| T4 DNA polymerase reaction mix | 20 microliters |
| Water | 10 microliters |

The reaction was incubated at 11° C. for 30 min. The reaction was subjected to phenol/chloroform extraction procedures and the DNA was recovered by ethanol precipitation. The precipitated pellet was dissolved in 10 μl water (to give a final concentration of 2 μg/μl).

The oligonucleotides were annealed to create a double-stranded linker, by mixing the following:

| | | |
|---|---|---|
| Oligonucleotide 1 (1 μg/μl) | 22.5 | μl |
| Oligonucleotide 2 (1 μg/μl) | 22.5 | μl |
| 10x annealing buffer | 5 | μl |
| Water | to 50 | μl |

The reaction was heated at 65° C. for 10 min; then allowed to cool at 15-25° C. for 2 h. The oligonucleotides in this protocol were designated 1 and 2.

The double-stranded linker was purified by column chromatography through a Sephadex G-50 spin column. The purified linker solution was concentrated by lyophilization to a concentration of 2 μg/μl.

Ligation of Linkers to Genomic DNA Fragments

The following reaction to ligate the linkers to genomic DNA fragments was set up. The reaction was incubated at 14° C. overnight.

| | | |
|---|---|---|
| Annealed linkers from Step (20 μg) | 10 | μl |
| Genomic DNA from Step (10 μl) | 5 | μl |
| T4 DNA ligase | 10 | U |
| 10x ligation buffer | 2 | μl |
| Water | to 20 | μl |

The reaction volume was adjusted to 500 μl with water and purify the ligated genomic DNA using a QIAquick PCR purification kit. The purified DNA was stored at a concentration of 1 μg/μl.

Primary Selection and Capture of Hybrids

To prepare the genomic DNA sample for hybridization to the microarray, linkered genomic DNA (10 μg) was resuspended in 3.5 μl of nuclease-free water and combined with 31.5 μl NimbleGen Hybridization Buffer (NimbleGen Systems Inc., Madison, Wis.), 9 μl Hybridization Additive (NimbleGen Systems), in a final volume of 45 μl. The samples were heat-denatured at 95° C. for 5 minutes and transferred to a 42° C. heat block.

To capture the target genomic DNA on the microarray, samples were hybridized to NimbleGen CGH arrays, manufactured as described, using a MAUI Hybridization System (BioMicro Systems, Inc., Salt Lake City, Utah) according to manufacturer instructions for 16 hours at 42° C. using mix mode B. (Singh-Gasson, S., et al., (1999), Maskless fabrication of light-directed oligonucleotides microarrays using a digital micromirror array. Nat. Biotechnol. 17:974-978; and Nuwaysir, E. F., et al., (2000), Gene expression analysis using oligonucleotide arrays produced by maskless photolithography, Genome Res. 12:1749-1755, incorporated by reference herein in its entirety). Following hybridization, arrays were washed twice with Wash Buffer I (0.2×SSC, 0.2% (v/v) SDS, 0.1 mM DTT, NimbleGen Systems) for a total of 2.5 minutes. Arrays were then washed for 1 minute in Wash Buffer II (0.2×SSC, 0.1 mM DTT, NimbleGen Systems) followed by a 15 second wash in Wash Buffer III (0.05×SSC, 0.1 mM DTT, NimbleGen Systems).

To elute the genomic DNA hybridized to the microarray, the arrays were incubated twice for 5 minutes in 95° C. water. The eluted DNA was dried down using vacuum centrifugation.

Amplification of the Primary Selected DNA

The primary selected genomic DNA was amplified as described below. Ten separate replicate amplification reactions were set up in 200 μl PCR tubes:

Reagents

| | | |
|---|---|---|
| Template: primary selection | 5 | μl |
| material | | |
| Oligonucleotide 3 (200 ng/μl) | 1 | μl |
| dNTPs (25 mM each) | 0.4 | μl |
| 10x PfuUltra HF DNA polymerase | 5 | μl |
| Reaction buffer | | |
| PfuUltra HF DNA polymerase | 2.5 | U |
| Water | to 50 | μl |

The reactions were amplified according to the following program:

| Cycle number | Denaturation | Annealing | Polymerization |
|---|---|---|---|
| 1 | 2 min at 95° C. | | |
| 2-31 | 30 s at 95° C. | 30 s at 55° C. | 1 min at 72° C. |

The reaction products were analyzed by agarose gel electrophoresis. The amplification products were purified using a QIAquick PCR purification kit. The eluted samples were pooled and the concentration of amplified primary selected DNA was determined by spectrophotometry. A volume of DNA in the pool equivalent to 1 μg was reduced to 5 μl in a speed vacuum concentrator. 1 μl (at least 200 ng) of the primary selected material was set aside for comparison with the secondary selection products. As necessary, subsequent rounds of enrichment were performed by further rounds of array hybridization and amplification of the eluted sample.

Preparation of Target Oligonucleotide Probes for Release from Microarray and Immobilization on Support

Probes were synthesized on a microarray, then were released using a base-labile Fmoc (9-fluorenylmethyloxycarbonyl) group. The probes were then immobilized onto the surface of a solid support using known methods for covalent or non-covalent attachment. Optionally, prior to immobilization onto the solid support, the synthesized probes were amplified using ligation mediated PCR, Phi29 or other amplification strategy to increase the amount of the synthesized probes. This material can now be used for direct sequencing, array based resequencing, genotyping, or any other genetic analysis targeting the enriched region of the genome.

It is understood that certain adaptations of the invention described in this disclosure are a matter of routine optimization for those skilled in the art, and can be implemented without departing from the spirit of the invention, or the scope of the appended claims.

1. A method of reducing the genetic complexity of a population of genomic nucleic acid molecules, the method comprising the steps of:
exposing a sample that comprises fragmented, denatured genomic nucleic acid molecules to a set of at least two oligonucleotide probes immobilized on a substrate under hybridizing conditions to capture target nucleic acid molecules that hybridize to the at least two probes, the at least two probes being characterized by normalized capture performance with regard to relative probe quantity;
separating unbound and non-specifically bound nucleic acids from the captured molecules; and
eluting the captured molecules from the substrate in an eluate pool having reduced genetic complexity relative to the sample.

2. The method of claim 1 further comprising the step of amplifying the eluted target nucleic acids.

3. The method of claim 1 wherein the probes are immobilized on the substrate by synthesizing the probe on the substrate.

4. The method of claim 3 wherein the substrate is a microarray.

5. The method of claim 4 wherein the probes are synthesized on the microarray using a maskless array synthesizer.

6. The method of claim 1 wherein the probes are immobilized on the substrate by synthesizing the probes and then applying the probes to the substrate.

7. The method of claim 6 wherein the substrate is a microarray.

8. The method of claim 6 wherein the substrate is selected from the group consisting of glass, metal, ceramic and polymeric beads.

9. The method of claim 8 wherein the probes are immobilized on the substrate by synthesizing the probes on a microarray, releasing the probes from the microarray and immobilizing the released probes on the substrate.

10.-11. (canceled)

12. The method of claim 1 wherein the normalized capture performance is achieved by a method comprising the steps of:
ascertaining the capture fitness of probes in the probe set; and
adjusting the quantity of at least one probe on the substrate.

13. The method of claim 1 wherein the normalized capture performance is achieved by a method comprising the steps of:
ascertaining the capture fitness of probes in the probe set; and
adjusting at least one of the sequence, the melting temperature and the probe length of at least one probe on the substrate.

14. The method of claim 1 wherein the normalized capture performance is achieved by a method comprising the steps of:
exposing the eluted target nucleic acids to the probes on the substrate under less stringent conditions than in the first exposing step such that the probes are saturated;
washing unbound and non-specifically bound nucleic acids from the substrate; and
eluting the bound target nucleic acids from the substrate.

15. The method of claim 1 wherein the normalized capture performance is achieved by a method comprising the steps of:
denaturing the eluted target nucleic acids to a single-stranded state;
re-annealing the single-stranded target nucleic acids until a portion of the target nucleic acids are double-stranded; and
discarding the double-stranded target nucleic acids and retaining the single stranded target nucleic acids.

16. The method of claim 1 wherein the probes hybridize to a genomic region of interest on nucleic acid fragments in the sample.

17. The method of claim 1 wherein the probes hybridize to sequences on target nucleic acid fragments comprising a genomic region of interest, the hybridizing sequences being separate from the genomic region of interest.

18. The method of claim 1 further comprising the step of performing at least a second hybridization step using at least one oligonucleotide probe related to but distinct from the probes used in the initial hybridization.
